(19) United States
(12) Patent Application Publication
(10) Pub. No.: US 2026/0174585 A1
(43) Pub. Date: Jun. 25, 2026

(54) SEAL PRESS FOR OSTOMY BARRIER ATTACHMENT

(71) Applicant: Northview Design LLC, Minnetrista, MN (US)

(72) Inventor: Darryl T. WROLSON, Minnetrista, MN (US)

(21) Appl. No.: 19/317,945

(22) Filed: Sep. 3, 2025

Related U.S. Application Data
(60) Provisional application No. 63/737,301, filed on Dec. 20, 2024.

(51) Int. Cl. A61F 5/443; A61F 5/44

(52) U.S. Cl. CPC A61F 5/443; A61F 5/4404

(57) ABSTRACT

A system for attaching an ostomy assembly to a patient is provided. The system includes an ostomy barrier. A seal press is configured to be disposed over the ostomy barrier and be temporarily coupled to the ostomy barrier. The seal press includes a sealing surface and a pressure surface. The sealing surface is detachably couplable to the ostomy barrier and the pressure surface is configured to receive and transfer a force to the ostomy barrier, such that the sealing element of the ostomy barrier attaches to a user.

### CROSS-REFERENCE TO RELATED APPLICATION

The present application is based on and claims the benefit of U.S. provisional patent application 63/737,301, filed Dec. 20, 2024, the content of which is hereby incorporated by reference in its entirety.

### FIELD OF THE DESCRIPTION

The present description relates to devices for assisting in an application of medical systems. More specifically, the present description relates to a device to assist in the attachment of an ostomy system.

### BACKGROUND

A wide range of medical bag systems exist for collecting bodily waste. Examples of such include ileostomy bags, colostomy bags, and urostomy bags. These bags can be collectively referred to as ostomy bags or ostomy pouches. Typically, an ostomy bag is attached to an area of a patient by a barrier which often includes some form of sealing component. In one example, the ostomy barrier includes a sealing component that contacts the skin of the patient to form the seal. The ostomy bag and ostomy barrier are collectively referred to as an ostomy system.

The discussion above is merely provided for general background information and is not intended to be used as an aid in determining the scope of the claimed subject matter.

### SUMMARY

A system for attaching an ostomy assembly to a patient is provided. The system includes an ostomy barrier having a barrier first side and a barrier second side. The barrier first side has an interface and the barrier second side has a sealing element. A seal press is configured to be disposed over the ostomy barrier and be temporarily coupled to the ostomy barrier. The seal press includes a sealing surface and a pressure surface. The sealing surface is detachably couplable to the barrier first side and the pressure surface is configured to receive and transfer a force to the ostomy barrier, such that the sealing element of the ostomy barrier attaches to a user.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

FIG. 1 is a partial pictorial, partial schematic illustration of an example of an ostomy system which includes an ostomy bag and an ostomy barrier.

FIG. 2A is a top plan view of a barrier first side of an ostomy barrier.

FIG. 2B is a perspective view of an ostomy barrier.

FIG. 2C is a bottom plan view of a barrier second side of an ostomy barrier.

FIG. 3A is a perspective view of a seal press according to one example.

FIG. 3B is a top plan view of a seal press according to one example.

FIG. 3C is a bottom plan view of a seal press according to one example.

FIG. 3D is an elevated end view of a seal press according to one example.

FIG. 3E is an elevated side view of a seal press according to one example.

FIG. 4A is an elevated end view of a seal press according to one example.

FIG. 4B is an elevated end view of a seal press according to one example.

FIG. 5 is bottom plan view of a seal press according to one example.

FIG. 6 is a block diagram of a seal press according to one example.

FIG. 7 is a partial schematic, partial exploded view of an application process of an ostomy barrier using a seal press.

FIG. 8 illustrates a method for applying an ostomy system to a patient.

### DETAILED DESCRIPTION

For the purpose of promoting an understanding of the principles of the present disclosure, reference will now be made to the examples illustrated in the drawings, and specific language will be used to describe the same. It will nevertheless be understood that no limitation of the scope of the disclosure is intended. Any alterations and further modifications to the described devices, systems, methods, and any further application of principles of the present disclosure are fully contemplated as would normally occur to one skilled in the art to which the disclosure relates. In particular, it is fully contemplated that the features, components, and/or steps described with respect to one example can be combined with the features, components, and/or steps described with respect to other examples of the present disclosure.

As discussed above, an ostomy system is used to assist users (e.g., ostomy patients) having a medical condition that inhibits the ability to eliminate bodily waste through natural pathways. These medical conditions can necessitate the need for an alternate exit for bodily waste, for instance, a stoma. A stoma is a surgically created opening on the body (e.g., abdomen) that connects to part of the intestines or urinary system, allowing bodily waste to pass directly through the stoma. Typically, the stoma protrudes from the skin of a patient and is left exposed. Users with a stoma often rely on ostomy systems for bodily waste collection and management.

Traditionally, ostomy systems are coupled to patients and include, among other things, an ostomy barrier and an ostomy bag. In some examples, ostomy systems include a single component that combines an ostomy bag with an integrated ostomy barrier. In other example ostomy systems, the ostomy barrier and ostomy bag are separate components and are removably coupleable to one another. The ostomy barrier is connected to a target area of a patient (generally surrounding the stoma) and the ostomy bag is connected to the ostomy barrier for waste collection. The ostomy barrier is a flexible, skin-friendly base that couples (e.g., adheres to the skin around the stoma. To accommodate the stoma, the barrier can include a central passageway designed to fit around the stoma such that waste is directed into the ostomy bag while minimizing skin exposure. A coupling component (e.g., adhesive component) is typically used to secure the barrier to the skin, providing a seal. However, a patient can face several challenges in ensuring a secure seal to prevent leaks.

The process of securely coupling an ostomy system to a patient's skin is somewhat challenging due to the nature of ostomy systems. For example, an ostomy barrier is typically made of a thin, flexible material without any suitable grip component for a patient's hands and/or fingers. This makes it difficult for a patient to properly hold and position the ostomy barrier over a stoma and further makes it difficult to apply an even pressure over the ostomy barrier. Precise alignment of the ostomy barrier over the stoma is also difficult as the flexibility of the barrier causes the adhesive to adhere to unintended surfaces (i.e. fingers, clothing, etc.) and complicates proper positioning.

During application, the limited space between the stoma and the ostomy barrier makes it difficult to create a consistent and secure seal. The space between the stoma and the adhesive area of the barrier is often less than one inch or even less than a half an inch which makes it challenging to apply consistent, uniform pressure around the stoma. Additionally, the skin area surrounding the stoma (e.g. peristomal skin) includes natural folds and contours which complicates the application of an effective seal. These difficulties, and the natural contours of the skin, can lead to an improper seal which can result in leaks, skin irritation, and the need for more frequent barrier replacements.

In some examples, a user can heat the application site prior to applying an ostomy barrier to improve adhesion and achieve a more secure seal. Applying warmth can soften the ostomy barrier and can enhance the skin's receptivity to the barrier. The ostomy barrier can become more flexible with heat and can seal (e.g. adhere) more effectively with heat application. However, effectively applying direct heat to the application site can be challenging, especially due to the sensitivity caused by a recent surgical procedure. Additionally, excessive heat can cause discomfort for the patient and risks irritation, making this method difficult to apply effectively.

With current ostomy systems and attachment methods, patients may have to replace the barriers relatively frequently, such as every few days. This is because the current attachment methods result in inconsistent and ineffective sealing which may lead to premature leaks. The replacement frequency with current ostomy systems and attachment methods increases costs to the patient. These ongoing costs create a financial burden for patients, particularly when frequent replacements are needed due to challenges with adhesion or forming a secure seal. The expense of maintaining an ostomy system can therefore place a strain on personal finances and healthcare resources.

Therefore, disclosed herein is a device that addresses the challenges above. A device is provided that enhances the barrier application process by providing for a uniform pressure over the ostomy barrier to ensure a reliable and secure seal. The device, in examples disclosed herein, comprises a seal press that can be used during the ostomy barrier application process. The seal press is configured to assist a patient in applying a uniform pressure over the barrier as it is applied to the skin. The seal press can also act as an additional grip to allow a user to guide the barrier onto the stoma and ensure correct positioning of the barrier.

While various examples detailed herein proceed in the context of medical devices related to ostomy systems, it will be understood that the devices, systems, and methods described herein can be used in various other medical device applications, such as, but not limited to, medical device applications which require a sealing pressure.

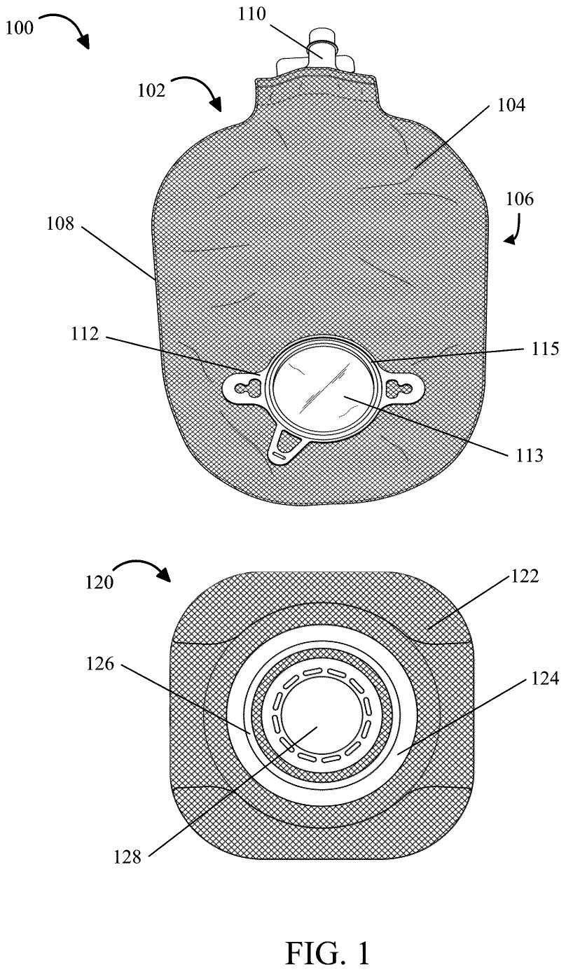

FIG. 1 is a partial pictorial, partial schematic illustration of an example of ostomy system 100 which includes ostomy bag 102 and ostomy barrier 120. Ostomy system 100 can be used for a wide variety of different applications such as, but not limited to, bodily waste management associated with a stoma. For example, a colostomy bag collects waste from the large intestine, an ileostomy bag handles waste from the small intestine, and a urostomy bag collects urine when the bladder is removed and/or bypassed. These bags enable effective bodily waste management for patients, such as patients with a stoma.

As illustrated in FIG. 1, ostomy bag 102 is in the form of a pouch designed to collect bodily waste when natural excretory functions are impacted by various medical conditions. Ostomy bag 102 can be manufactured from a durable, odor-resistant, multi-layered material to securely contain the waste and maintain adequate hygiene. Ostomy bag 102 has bag first side 104 and bag second side 106 (opposite the bag first side 104). In one example, bag first side 104 can be constructed of a skin-friendly material and bag second side 106 can be constructed of a more durable plastic material to prevent leakage from unintentional contact. In another example, bag first side 104 and bag second side 106 can be made of the same material. Additionally, bag first side 104 can be secured to second side 106 by coupling mechanism 108. In one example, coupling mechanism 108 is a leak-proof seal (e.g., stitch, etc.) that has a sufficient strength to hold the bag sides together to prevent leakage.

In one example, ostomy bag 102 includes a drainage outlet at one end. The configuration of the drainage outlet can vary based on the type of ostomy system needed (i.e. colostomy, urostomy, etc.). As shown in FIG. 1, ostomy bag 102 includes drainage outlet 110. Drainage outlet 110 allows a user to empty the bag's contents as needed without full replacement of ostomy bag 102 which increases usability and convenience. In one example, ostomy bag 102 can include a filter to release gases, which prevents the bag from ballooning and helps control odor. Additionally, ostomy bag 102 can include port 112. Port 112 can be an aperture that extends from the external side of ostomy bag 102 to the internal side of ostomy bag 102. Port 112 can include aperture 113 having an annular shape or any other shape. Additionally, port 112 can comprise a variety of shapes. In one example, port 112 can be integrated into bag first side 104 of ostomy bag 102 and function as a connection mechanism for connecting to a corresponding component (e.g., connection mechanism) of ostomy barrier 120. The connection between ostomy bag 102 and ostomy barrier 120 can provide a leakproof seal between ostomy bag 102 and ostomy barrier 120. Port 112 can include mating mechanism 115 for connection with barrier 120. In the example shown, port 112 includes a female mating feature configured to receive a male mating feature of barrier 120 but could, in other examples, be vice-versa or other forms of mating mechanisms.

Ostomy barrier 120 is also shown in FIG. 1. Ostomy barrier 120 includes barrier first side 122. Barrier first side 122 can be constructed from the same material as bag first side 104 to ensure a secure connection. Additionally, barrier first side 122 includes interface 124. Interface 124 can include a mating feature 126. In one example, mating feature 126 is a flange or other projection that extends outward from interface 126 (e.g. male mating feature). In one example, mating feature 126 can be a female mating feature. Interface 124 can be shaped and sized to align with port 112 of ostomy bag 102 to allow for a precise connection. When in use, mating feature 126 of ostomy barrier 120 engages with port 112 of ostomy bag 102, ensuring that the barrier and bag are properly mated and securely attached. Ostomy barrier 120 also includes central aperture 128 designed to fit around the stoma of a user.

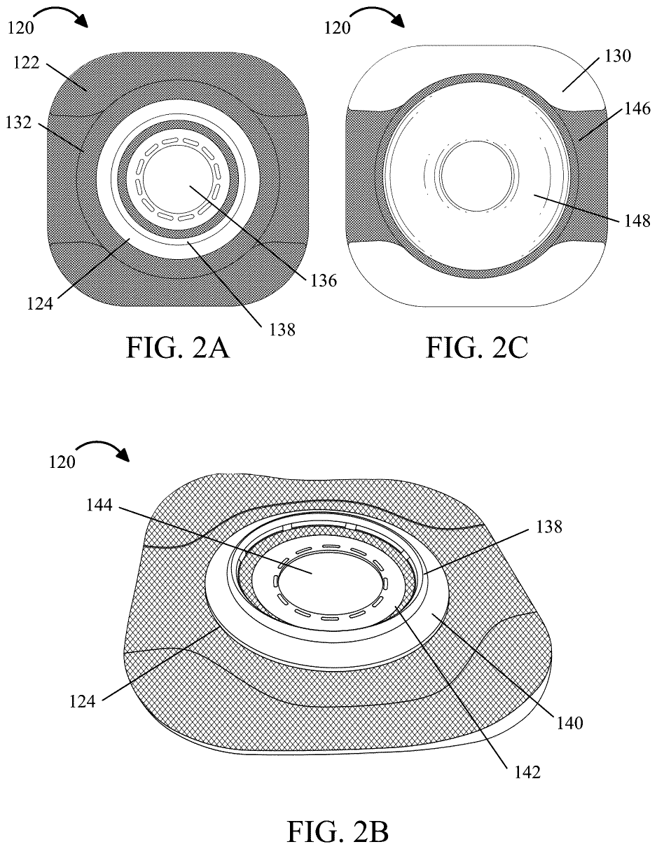

FIGS. 2A-2C are schematic illustrations of various views of ostomy barrier 120. Ostomy barrier 120 is a component of ostomy system 100 that can be constructed to couple to a user (e.g. adhere to the skin of a user) to create a base for the connection of ostomy bag 102. Ostomy barrier 120, and any included features, could be a wide variety of materials, shapes, or sizes. Additionally, the material composition can vary and include skin-friendly materials and/or flexible compounds that adapt to different skin types and provide an effective connection. Ostomy barrier 120 can include barrier first side 122 and barrier second side 130 (opposite barrier first side 122). In one example, barrier first side 122 and barrier second side 130 are made from the same material. In another example, barrier first side 122 and barrier second side 130 are made from different materials.

FIG. 2A is a top plan view of barrier 120 showing barrier first side 122. Barrier first side 122 can include surface 132 and interface 124. In one example, surface 132 is constructed from a flexible, skin-friendly material that is both soft and durable, such as hydrocolloid or silicone-based compounds. Ostomy barrier 120 also contains central opening 136 designed to accommodate a user's stoma, allowing for the direct flow of waste into the attached ostomy bag (e.g., 102). Central opening 136 can be pre-sized or can be customizable to fit around the stoma and minimize exposure to waste and reduce the risk of leaks. The shape of central opening 136 can vary in size and shape and can be varied to accommodate different stomas.

In the example shown, interface 124 is positioned around the central opening 136, such that interface 124 surrounds central opening 136. Interface 124 can include coupling feature 138 or any of a number of other coupling components (e.g. mating features). In the example shown, coupling component 138 is a flange (e.g., an annular flange). Interface 124 allows ostomy bag 102 to lock and/or snap into place, depending on the specific system design. In one example, the structure of coupling component 138 is designed to match the corresponding attachment (i.e. port 112) on ostomy bag 102 to form a tight seal that prevents leaks and ensures stability. Some barriers can include additional features, such as a convex shape for patients with recessed stomas or soft edges for added comfort.

FIG. 2B is a perspective view of ostomy barrier 120. FIG. 2B further illustrates interface 124 and coupling component 138, according to one example. As shown, interface 124 includes outer periphery 140 and inner periphery 142 separated by coupling component 138. Coupling component 138 can include a ridge and/or lip to assist in the connection with ostomy bag 102. In alternative examples, interface 124 can include a recessed slot to receive a connection element of an ostomy bag having a flange or projection. Ostomy barrier 120 includes central passage 144 which extends through ostomy barrier 120 to allow accommodation of a stoma of a patient.

FIG. 2C is a bottom plan view of ostomy barrier 120 showing barrier second side 130. In one example, barrier second side 130 is the side that faces and contacts the skin of a user. Barrier second side 130 can include surface 146. Surface 146 can include central opening 148. Central opening 148 can include any polygonal shape sufficient in surrounding, or partially surrounding, a stoma of a patient. Central opening 148 forms central passage 144 (as shown in FIG. 2B).

Surface 146 of barrier second side 130 can be made of the same material as or different than the material of surface 132 of barrier first side 122. Barrier second side 130 can also include a sealing element 148 that can be included as part of or coupled to barrier second side 130. In one example, sealing element 148 is applied to create a bond with the skin (e.g. peristomal skin) on a patient, such that ostomy barrier 120 is attached to the patient. In one example, sealing element 148 contacts the skin of an abdomen region of a patient, and adheres to this skin. Sealing element 148 can be an adhesive material to form a bond with the skin and therefore form a seal around the stoma. It is understood that sealing element 148 can be any material sufficient to adhere to a skin surface. The material can withstand moisture while minimizing skin irritation, as the barrier can need to be in place for several days at a time. In one example, sealing element 148 can be heat-assisted. In another example, sealing element 148 can be heat-activated. Applying a heat source to ostomy barrier 120 can increase the flexibility and adhesive properties of sealing element 148.

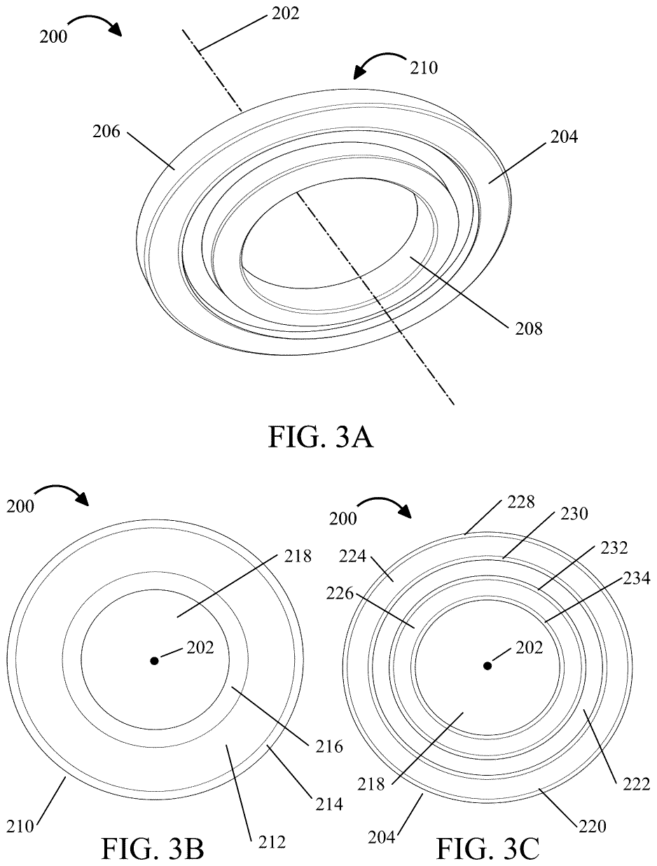

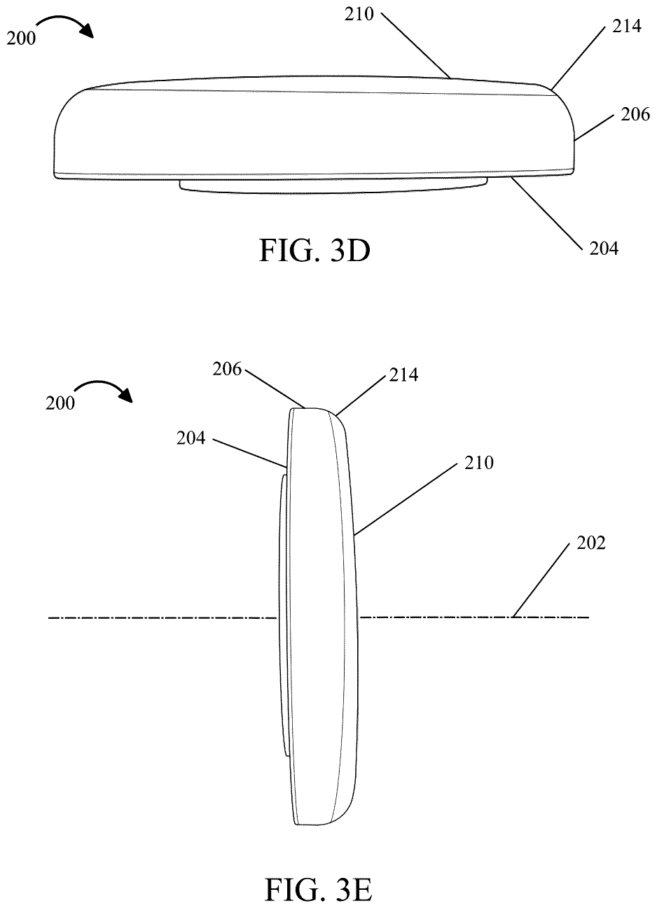

FIG. 3A is a perspective view of seal press 200. Seal press 200 assists in forming a seal between ostomy barrier 120 and the skin of a patient during an application of ostomy barrier 120. Seal press 200 can withstand a force applied by a user, such that it is capable of exerting a controlled pressure onto a surface (i.e. ostomy barrier 120). Therefore, in one example, seal press 200 is a pressure applicator that assists in applying a controlled and uniform pressure during the application of ostomy barrier 120.

As shown, seal press 200 is ring-shaped to accommodate or match with the design of an ostomy barrier (e.g., ostomy barrier 120) but can be other shapes such as to accommodate or match with other types of ostomy barriers. Seal press 200 can include a material sufficient to withstand the user-applied force. Such material can be composed of rubber, silicone, stainless steel, aluminum, plastic, recycled material, disposable material, or other material. The material composition of seal press 200 can include a single material or can include a combination of multiple materials. Seal press 200 can be manufactured through a variety of different processes such as injection molding or 3D printing, among others. As shown in FIG. 3A, seal press 200 can be symmetrical to central axis 202 and can be designed to revolve uniformly (or approximately uniformly) around central axis 202. It will be understood that the specific design considerations and material choices for seal press 200 can vary greatly, and the examples provided herein are not intended to limit the scope of the claimed subject matter.

In one example, seal press 200 can include base portion 204, external side wall 206, internal side wall 208, and top portion 210. Each feature will now be described in more detail below with regard to FIGS. 3B, 3C, 3D, and 3E.

FIG. 3B is a top plan view of seal press 200. As shown, seal press 200 includes top portion 210. In one example, top portion 210 includes top surface 212 which includes, in the illustrated example, a substantially flat surface area. Top surface 212 can have an annular shape forming an annular surface area. Top surface 212 is constructed to receive and withstand a force from a user (e.g., a force applied by a user's hand), such that the force is transferred through the seal press to affect a surface (e.g. surface 132 and/or surface 146) under the seal press. Top surface 212 can be constructed using a variety of textures or finishes to improve grip, depending on the intended application. For example, top surface 212 can include a matte finish, shallow ridges, and/or grips to increase grasp.

Top surface 212 can have an outer periphery 214 and an inner periphery 216 at edges connecting top surface 212 to each respective side wall (i.e. external side wall 206 and internal side wall 208). External side wall 206 and internal side wall 208 can extend from top portion 210 to base portion 204 in a parallel direction in regard to central axis 202. Alternatively, each side wall can taper away from or towards central axis 202. Thus, each side wall can be straight or curved.

In one example, outer periphery 214 can have a first circumference and inner periphery 216 can have a second circumference, forming an annulus. Within inner periphery 216, there can be central aperture 218 spanning the perimeter of internal side wall 208 of seal press 200, forming a ring-like shape. Each periphery can form a boundary of top portion 210, which connects top portion 210 with each respective side wall. These boundaries can include a contour such as a right angle, beveled edge, or other contoured shape.

FIG. 3C is a bottom plan view of seal press 200. As shown, seal press 200 includes base portion 204, base surface 220, and recess 222. Base portion 220 can form a surface feature that is sufficient to be disposed over a target surface, such that base portion 204 can apply a pressure to the target surface. Similar to top surface 212, base surface 220 can include a surface area. In one example, base surface 220 includes outer surface 224 and inner surface 226, each having its own respective circumference. Outer surface 224 can include a circumference greater than the circumference of inner surface 226, with regard to central axis 202. As shown, outer surface 224 and inner surface 226 can each form an annulus separated by a defined space (i.e. recess 222). It is understood that the shape and/or contour of the illustrated example is not intended to limit the scope of the present disclosure, as any of a variety of design configurations can be employed.

Outer surface 224 can include external side 228 and internal side 230. Additionally, inner surface 226 can include external side 232 and internal side 234. The defined space separating outer surface 224 and inner surface 226 can create a separation between internal side 230 of outer surface 224 and external side 232 of inner surface 226. In one example, the defined space can be recess 222. Recess 222 can include a gap, slot, channel or other similar depression. In one example, recess 222 is formed as a depression or channel within base surface 220. It is understood that recess 222 can vary in shape, depth, width, and orientation, depending on the design requirements of seal press 200 for a given application (e.g., to match the barrier).

Recess 222 can be configured to separate external side wall 206 from internal side wall 208. In one example, recess 222 can include a continuous channel running along a perimeter between external side wall 206 and internal side wall 208. Recess 222 can also span across a circumference between outer surface 224 and inner surface 226. Additionally, the edges of recess 222 can be straight, beveled, or rounded, depending on the intended interaction with additional components. Recess 222 can be constructed to serve as a receptacle for another component (e.g. coupling component 138 of ostomy barrier 120). In such examples, the width of recess 222 can be designed to fit snugly around the additional component, preventing undesired movement. Recess 222 can include an internal side wall, and the internal side wall can have a securing element, such as a ridge or lip, to receive an additional component. In an alternative example, recess 222 is not a depression and instead protrudes from base surface 220, such that a protruding component is formed. In one example, the protruding component is a flange. Therefore, seal press 200 can be inserted into a recess of an additional component (e.g. ostomy barrier 120) to form a lock and key configuration.

FIG. 3D is an elevated end view of seal press 200. Similarly, FIG. 3E is an elevated side view of seal press 200. FIGS. 3D and 3E contain similar features. Accordingly similar features are numbered accordingly and are discussed below in unison. As discussed above, seal press 200 can include top portion 210, outer periphery 214, external side wall 206, and base portion 204. Outer periphery 214 can be a portion of top portion 210 and can have a beveled edge, as discussed above with regard to FIG. 3A. External side wall 206 extends from top portion 210 to base portion 204 forming a collective body portion of seal press 200. As shown in FIGS. 3D and 3E, internal side wall 208 can extend below the plane of base portion 204. Alternatively, internal side wall 208 can align with the plane of base portion 204, or not reach the plane of base portion 204 at all.

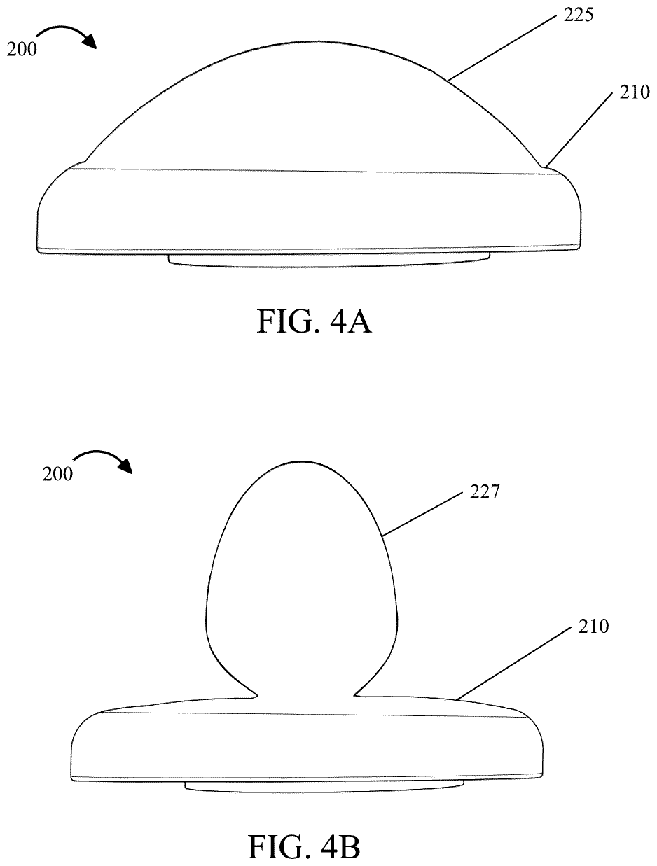

Alternatively, in one example, seal press 200 does not form an annulus at all. For instance, top portion 210 does not include central aperture 218. In one example, top portion 210 can instead include a handle or similar grip-enhancing component (examples shown in FIGS. 4A and 4B).

As shown in FIG. 4A, seal press 200 includes protrusion 225. Protrusion 225 is one example of a grip-enhancing component. Protrusion 225 can be a bump or dome component that covers and protects a stoma of a patient. Protrusion 225 can be a raised structure that extends from top portion 210 for increased grip and precision. Additionally, protrusion 225 can form a small knob, tab, ridge, or other grip component.

As shown in FIG. 4B, seal press 200 can include handle 227. Handle 227 is one example of a grip or other grip-enhancing component. Handle 227 on top portion 210 can provide a firm point for a user to press which ensures uniform pressure distribution. Protrusion 225 and/or handle 227 can include a variety of different materials to enhance grip and precision during application. It will be understood that any grip-enhancing component (e.g. protrusion) on seal press 200 would be sized to accommodate a stoma of a patient and be compatible with the application of the ostomy system.

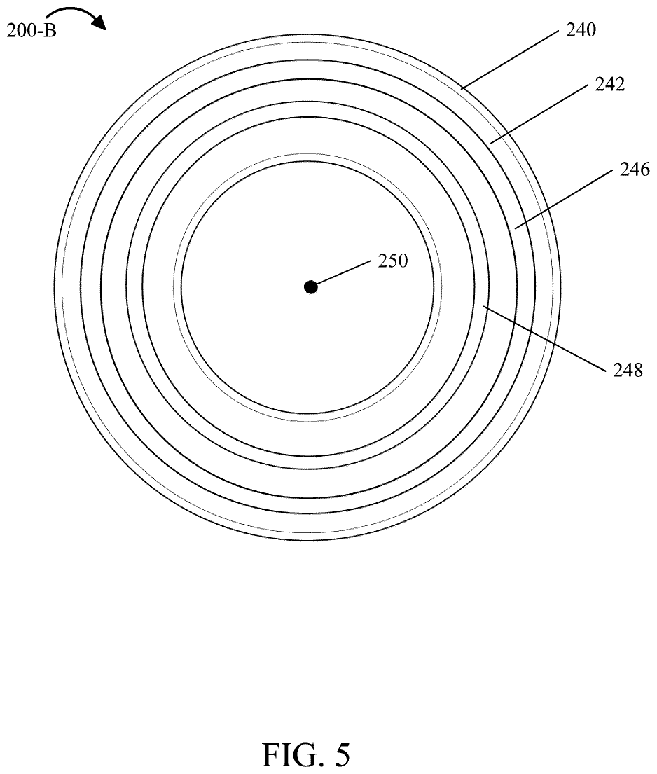

FIG. 5 is a bottom plan view of seal press 200-B according to one example. It is understood that seal press 200-B is similar to seal press 200 and can include similar structural characteristics as seal press 200. As shown, seal press 200-B is similar to seal press 200 except that seal press 200-B includes multiple recesses (e.g. recess 246 and recess 248), at different diameters, configured to engage with different barriers. In this way, a singular seal press can be used with different types of barriers (e.g., barriers of different sizes and/or having differently sized interfaces). As shown, seal press 200-B includes base portion 240, base surface 242, and recess 246. Base portion 240 can form a surface feature that is sufficient to be disposed over a target surface, such that base portion 204 can apply a pressure to the target surface. Similar to top surface 212 of seal press 200, base surface 242 includes a surface area.

Additionally, base surface 242 also includes recess 248. In one example, recess 246 and recess 248 are disposed on the surface area of base surface 242. As shown, recess 246 and recess 248 are positioned at different diameters relative to central axis 250. As shown, recess 246 has a first width, while recess 248 has a second width, different from the first width of recess 246. Alternatively, recess 246 and recess 248 can have the same width. Recess 246 and/or recess 248 can include a gap, slot, channel or other similar depression. It is understood that recess 246 and recess 248 can vary in shape, depth, width, and orientation, depending on the design requirements of seal press 200-B for a given application (e.g., to match the barrier).

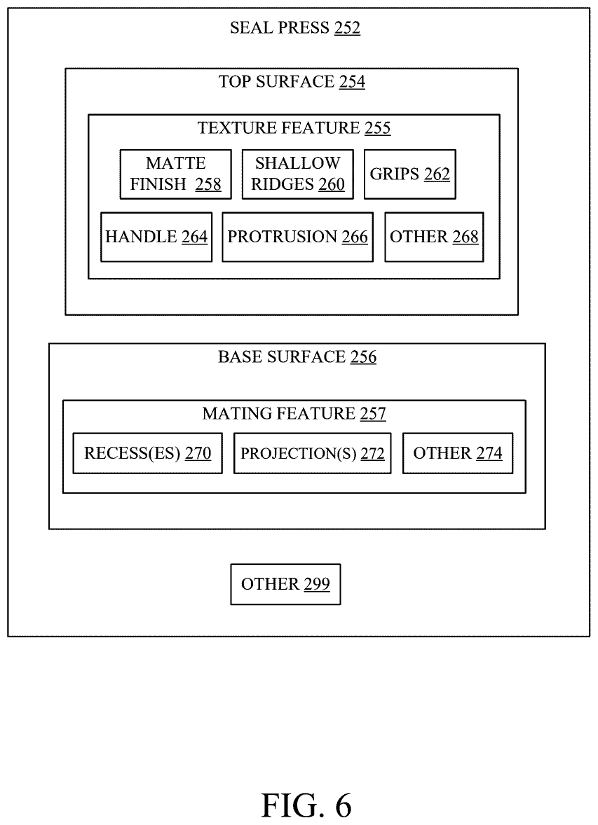

FIG. 6 is a block diagram of seal press 252 according to one example. In one example, seal press 252 is seal press 200 or 200-B, as discussed above. As shown, seal press 252 comprises top surface 254 and base surface 256. In one example, top surface 254 includes texture feature 255. Texture feature 255 is used to provide the user with tactile components for increased grip and secure handling. In one example, texture feature 255 includes providing matte finish 258 on top surface 254. In another example, texture feature 255 is shallow ridges 260 or grips 262. Shallow ridges 260 and grips 262 are elevated features (i.e. repeated bumps) for enhanced handling. In another example, texture feature 255 is handle 264 or protrusion 266. Handle 264 and protrusion 266 are structural components that project from top surface 254 and provide a defined grasp point for a user. In one example, protrusion 266 is similar to or the same as protrusion 225 of seal press 200 as shown in FIG. 4B. In one example, handle 264 is similar to or the same as handle 227 of seal press 200 as shown in FIG. 4B. In other examples, texture feature 255 can include other features represented by other 268.

Base surface 256 includes mating feature 257. In one example, mating feature 257 includes one or more recesses 270. Recesses 270 are configured to receive a coupling component (e.g., 138) of an ostomy barrier (e.g., 120). In one example, base surface 256 includes only a single recess 270. The single recess, in one example, is similar to or the same as recess 222. In another example, base surface 256 can include multiple recesses 270. For example, base surface 256 can include two recesses 270. The two recesses can be similar to or the same as recess 246 and recess 248. Alternatively, mating feature 257 can include projection(s) 272. In one example, projection(s) 272 replaces recesses 270 and forms a structure that extends outward from base surface 256. Projection(s) 272 is a male mating feature for application with an associated ostomy barrier (e.g., an ostomy barrier having a female mating feature). In other examples, mating feature(s) 257 can include other features represented by other 274.

Seal press 252 can include various other items 299 as well, such as other items described in FIGS. 1-5.

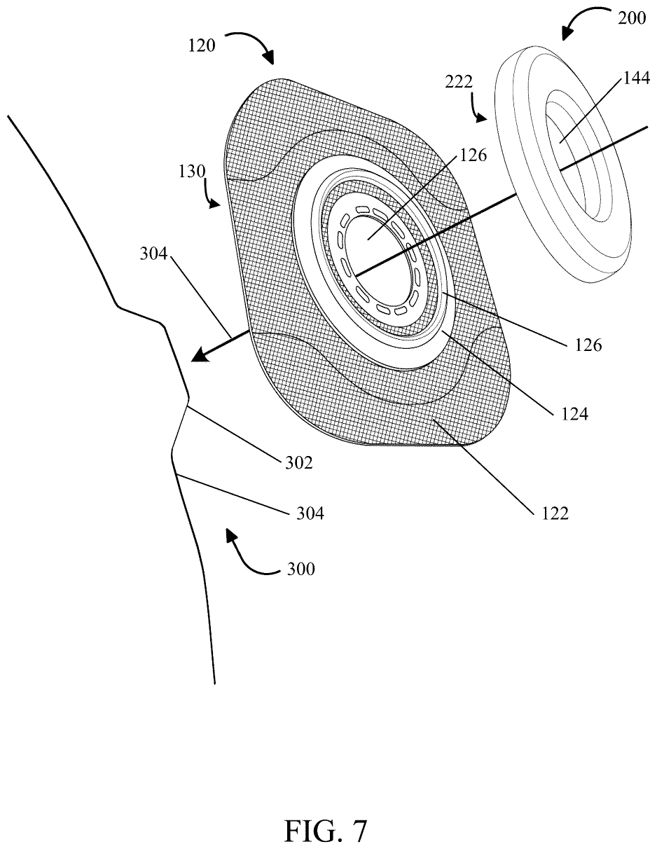

FIG. 7 is a partial schematic, partial exploded view of an application process of ostomy barrier 120 using seal press 200. FIG. 5 illustrates ostomy barrier 120 and seal press 200. As shown, ostomy barrier 120 can be applied to target area 300, which, in the illustrated example, is located around the abdomen region of a patient. Target area 300 can also include stoma 302. As shown, target area 300 includes stoma 302 and peristomal skin 304.

As discussed above, ostomy barrier 120 has barrier first side 122 and barrier second side 130. Barrier second side 130 contacts the skin and is applied to the skin (e.g. peristomal skin) around stoma 302 of the patient. Barrier first side 122 includes interface 124 with mating feature 126. In one example, ostomy barrier 120 is positioned on the patient before seal press 200 is pressed onto target area 300. Then, as seal press 200 is pressed onto ostomy barrier 120, base portion 204 of seal press 200 contacts barrier first side 122 and thus provides a sealing force on barrier second side 130, such that barrier second side 130 adheres to target area 300 and forms a seal.

In some instances, the positioning of ostomy barrier 130 on target area 300 can be difficult for some patients given the flexible nature of the barrier. Therefore, seal press 200 can be coupled to ostomy barrier 120 prior to the application to target area 300 to provide for easier application of barrier to the target area (e.g. provide a user with enhanced grip). Interface 124 of ostomy barrier 120 can be configured to be mated with base portion 204 of seal press 200. More specifically, in one example, coupling component (e.g., flange) 138 of interface 124 is received by recess 222 of base portion 204 of seal press 200. This connection can be temporary, as seal press 200 can be removed from ostomy barrier 120, such as after an ostomy barrier application process is complete (e.g., after establishing seal between the barrier and the skin). In one example, after the seal press 200 is removed, ostomy bag 102 can be attached to ostomy barrier 120.

Once seal press 200 is connected to ostomy barrier 120, a user can use seal press 200 to guide ostomy barrier 120 into a correct position over stoma 302, such that the seal press 200 and ostomy barrier 120 function as a single unit. In one example, this single unit is referred to as ostomy press 310. As would be appreciated by one skilled in the art, central aperture 128 of barrier 120 and central passage 144 of seal press 200 are aligned to accommodate stoma 302. In one example, the correct positioning includes guiding central aperture 128 of ostomy barrier 120 over stoma 302. After achieving correct placement, a user can apply a force to seal press 200 in the direction towards target area 300, along arrow 304. This force can be exerted by a user's hand and/or fingers to provide a more constant and uniform pressure across the surface area of top portion 210 of seal press 200.

Seal press 200 provides more evenly distributed pressure, which is beneficial for proper attachment of ostomy barrier 120 and for establishing a robust seal. The even distribution of pressure helps apply a uniform pressure over ostomy barrier 120. Additionally, the even distribution of pressure provided by seal press 200 helps smooth and evenly flatten the natural contours of the skin (e.g. peristomal skin). This creates a more effective application site for the attachment of an ostomy system. Thus, the even pressure distribution from seal press 200 reduces the risk of premature leak pathways caused by the interaction between the ostomy barrier and the skin's natural irregularities.

As the force is applied to seal press 200, ostomy barrier 120 is compressed between target area 300 and seal press 200. As discussed above, second side 130 of ostomy barrier 120 includes sealing element 148. The applied force to seal press 200 enhances the effective sealing action of seal element 148 as it contacts target area 300. The result is a uniform seal around stoma 302 that minimizes gaps between seal element 148 and target area 300, which minimizes the risk for leakage. Once an effective seal is achieved, seal press 200 can be removed from ostomy barrier 120, such that ostomy bag 102 can be attached to barrier 120. Accordingly, the improved seal provided by seal press 200 minimizes leaks and increases longevity of ostomy barrier 120, thus improving patient comfort and reducing costs.

In one example, seal press 200 can be heated prior to the connection to ostomy barrier 120. Alternatively, seal press 200 and ostomy barrier 120 can be heated together before being positioned on target area 300. As discussed above, a heat source can assist the application of ostomy barrier 120 to target area 300. Seal press 120 can be heated to a desired temperature, such as a temperature above the average human external body temperature.

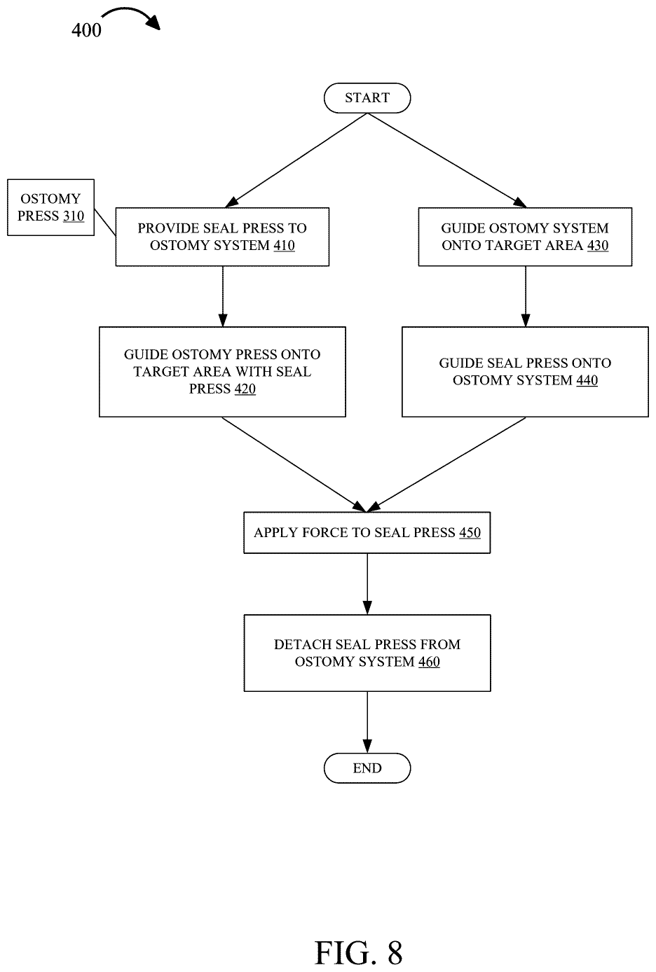

FIG. 8 illustrates a method for applying an ostomy system to a patient. In one example, an ostomy system is a singular unit that includes the ostomy bag and ostomy barrier coupled together. In another example, the ostomy system includes two separate components, where the ostomy bag and ostomy barrier are detachably couplable. As would be appreciated by one skilled in the art, a seal press, such as seal press 200, can be designed to accommodate both configurations. Method 400 describes an application process of applying an ostomy system to a target area of a patient (i.e. target area 300). Method 400 begins with either step 410 or step 430. Step 410 includes providing seal press 200 to an ostomy system. In one example, the ostomy system includes separate components and ostomy barrier 120 is separate from ostomy bag 102. In this example, seal press 200 is detachably coupled to ostomy barrier 120 to form ostomy press 310. Then in step 420, seal press 200 is used to guide ostomy press 310 onto the target area to align ostomy barrier 120. Alternatively, a user can start with step 430. Step 430 illustrates that an ostomy system is guided directly onto the target area before seal press 200 is provided. In one example, ostomy barrier 120 is guided onto the target area. Then, in step 440, seal press 200 is guided onto the ostomy system which is positioned on the target area.

After completion of either step 420 or step 440, the method converges at step 450 where force is applied to seal press 200 to secure ostomy system onto the target area and to form a seal. A force is applied from a user through contact from their hands and/or fingers. The force can be a uniform pressure directed towards the target area, as shown by arrow 304 in FIG. 5. Next, in step 460, seal press 200 is detached from ostomy system, completing the attachment process of ostomy system and establishment of the seal. In one example, where the ostomy system includes separate components, after seal press 200 is detached, an ostomy bag, such as ostomy bag 102, can be attached to ostomy barrier 120. Overall, method 400 improves the seal of an ostomy system by assisting in precise alignment, uniform pressure application (i.e. pressure application in a uniform manner), and secure attachment to the target area.

In summary, various embodiments and examples for systems and methods for applying an ostomy system to a patient have been disclosed. Although the systems and methods for applying the ostomy system have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

What is claimed is:

1. A system for attaching an ostomy assembly to a user, the system comprising:
an ostomy barrier comprising a barrier first side and a barrier second side, the barrier first side having a coupling interface, and the barrier second side having a sealing element;
a seal press configured to be disposed over the ostomy barrier, the seal press comprising a seal surface and a pressure surface;
wherein the seal surface is detachably couplable to the coupling interface of the barrier first side; and
wherein the pressure surface is configured to exert a force on the ostomy barrier, such that the sealing element attaches to a user.

2. The system of claim 1, wherein the seal press is detached from the ostomy barrier.

3. The system of claim 2, and further comprising an ostomy bag, wherein the ostomy bag is coupled to the interface of the ostomy barrier.

4. The system of claim 1, wherein the interface comprises an annular flange.

5. The system of claim 4, wherein the sealing surface comprises a recess configured to receive the annular flange.

6. The system of claim 1, wherein the force exerted by the seal press is configured to flatten a target area on a user.

7. The system of claim 1, wherein the barrier second side comprises an annular surface area.

8. The system of claim 7, wherein the annular surface area comprises an aperture to surround a stoma of a user.

9. The system of claim 8, wherein the pressure surface of the seal press is configured to exert the force across the annular surface area in a uniform manner.

10. The system of claim 1, wherein the sealing element is an adhesive element.

11. A method of applying an ostomy system to a user, the method comprising:
connecting a seal press to an ostomy barrier to form an ostomy press;
positioning the ostomy press on a user;
applying a force to the ostomy press, the force being directed toward the user;
detaching the seal press from the ostomy barrier; and
coupling an ostomy bag to the ostomy barrier.

12. The method of claim 11, wherein the applied force is a uniform pressure.

13. The method of claim 11, wherein the ostomy barrier has a barrier first side and a barrier second side.

14. The method of claim 13, wherein the barrier first side is received by the seal press.

15. The method of claim 13, wherein the barrier second side comprises a sealing element.

16. The method of claim 11, wherein the seal press is heated prior to being connected to the ostomy barrier.

17. The method of claim 11, wherein the seal press is configured to flatten a target area on a user.

18. A seal press for ostomy system attachment, the seal press comprising:
a top portion having an outer periphery and an inner periphery;
a base portion forming a base surface, the base surface having an outer surface and an inner surface separated by a space;
an external wall extending from the outer surface of the base portion to the outer periphery of the top portion; and
an internal wall extending from the inner surface of the base portion to the inner periphery of the top portion.

19. The seal press of claim 18, wherein a handle component is disposed on the top portion.

20. The seal press of claim 18, wherein the space comprises a recessed channel.
